SEQUENCE LISTING (3 sequences)

SEQ ID NO: 1 - protein, 25 aa, synthetic construct
  source 1..25: note = derived from human Serum Amyloid P (SAP) protein regions that are not homologous to C-reactive protein (CRP
KERVGEYSLYIGRHKVTSKVIEKFP

SEQ ID NO: 2 - protein, 12 aa, synthetic construct
  source 1..12: note = derived from human Serum Amyloid P (SAP) protein regions that are not homologous to C-reactive protein (CRP)
ILSAYQGTPLPA

SEQ ID NO: 3 - protein, 11 aa, synthetic construct
  source 1..11: note = derived from human Serum Amyloid P (SAP) protein regions that are not homologous to C-reactive protein (CRP
IRGYVIIKPLV

(19) United States
(12) Patent Application Publication
(10) Pub. No.: US 2026/0115201 A1
(43) Pub. Date: Apr. 30, 2026

(54) POTENTIAL THERAPEUTICS FOR FIBROSIS BY BLOCKING THE INCREASE OF A KEY ENZYME

(71) Applicant: THE TEXAS A&M UNIVERSITY SYSTEM, COLLEGE STATION, TX (US)

(72) Inventors: RICHARD GOMER, COLLEGE STATION, TX (US); DEBORAH BELL-PEDERSEN, COLLEGE STATION, TX (US); WENSHENG CHEN, CHANGSHA,HUNAN (CN)

(21) Appl. No.: 19/120,870

(22) PCT Filed: Oct. 12, 2022
(86) PCT No.: PCT/US2022/077935

§ 371 Date: Apr. 14, 2025

(51) Int. Cl. A61K 31/551; A61K 9/00; A61K 45/06; A61P 11/00; A61P 43/00

(52) U.S. Cl. CPC A61K 31/551; A61K 9/0019; A61K 45/06; A61P 11/00; A61P 43/00

(57) ABSTRACT

The subject invention pertains to compounds and compositions comprising one or more inhibitor of DDX3 and methods for suppressing fibrosis by administration of one or more DDX3 inhibitor to a subject or in an in vitro setting.

### STATEMENT REGARDING FEDERALLY SPONSORED RESEARCH

This invention was made with government support under R01HL132919 awarded by the National Institutes of Health. The government has certain rights in the invention.

### STATEMENT REGARDING SEQUENCE LISTING

The Sequence Listing for this application is labeled “Seq-List.xml” which was created on Sep. 12, 2022 and is 4,071 bytes. The entire content of the sequence listing is incorporated herein by reference in its entirety.

### BACKGROUND OF THE INVENTION

The cellular ATPase/RNA helicase X-linked DEAD-box polypeptide 3 (DDX3) is a human protein involved in several biological functions such as RNA metabolism (transcription, splicing, mRNA nuclear-cytoplasmatic export, translation), ribosome biogenesis, cell cycle regulation, apoptosis, Wnt-β-catenin signaling, and anti-viral innate immune signaling pathways. DDX3 has been identified as a target for the development of new drugs against viruses and neoplastic diseases.

### BRIEF SUMMARY OF THE INVENTION

The present disclosure relates to compounds and compositions comprising one or more inhibitors of DDX3 and methods for suppressing the pro-fibrosis effects of factors such as TGF-β1 or IL-6 by administration of one or more DDX3 inhibitor to a subject or in an in vitro setting.

### BRIEF DESCRIPTION OF THE DRAWINGS

FIGS. 1A-1B are graphs quantifying the NEU3 protein detected in human small airway epithelial cells (HSAEpC) by western blots. FIG. 1A quantifies NEU3 in TGF-β1 treated HSAEpC. FIG. 1B quantifies NEU3 in IL-6 treated HSAEpC. Points indicate individual values for control (−), TGF-β1 or IL-6 treated cells, control with RK33 or IN-1, TGF-β1 or IL-6 treated with RK33 or IN-1, respectively. Values are mean±SEM, n=3. **p<0.01, ***p<0.001 vs control, ^{##}p<0.01, ^{#}p<0.001 vs TGF-β1 (FIG. 1A) or IL-6 (FIG. 1B) treated cells (1-way ANOVA, Dunnett's test).

FIGS. 2A-2C are western blots and graphs quantifying DDX3 and NEU3 protein detected in human lung fibroblast (HLF) by western blots. FIG. 2A shows the images of DDX3 and NEU3 western blots, and a Coomassie-stained gel of the samples. Molecular masses in kDa of molecular mass standards are at the left of the image of the Coomassie-stained gel. FIG. 2B quantifies DDX3 in DDX3-siRNA treated HLF. FIG. 2C quantifies NEU3 in DDX3-siRNA treated HLF. Points indicate individual values for non-transfected (NT), control-siRNA transfected (C-si), DDX3-siRNA transfected (D-si), in the absence (left 3 bars) and presence (right 3 bars) of TGF-β1. Values are mean±SEM, n=3. **p<0.01, ***p<0.001 (1-way ANOVA, Dunnett's test).

FIG. 3 is a graph quantifying the survival of mice. Curves indicate percent survival values for saline with buffer (S+B), saline with RK33 (S+RK), bleomycin with buffer (BL+B), and bleomycin with RK33 (BL+RK), respectively. n=5 mice for S+B, S+RK, and BL+RK, and n=8 mice for BL+B. **p<0.01 vs BL+B (Mantel-Cox test).

FIG. 4 is a graph quantifying the weight changes of mice. Points indicate average values as a percent of the day 0 weight for saline with buffer (S+B), saline with RK33 (S+RK), bleomycin with buffer (BL+B), and bleomycin with RK33 (BL+RK), respectively. Error bars indicate SEM. n=5 mice for S+B, S+RK, and BL+RK. n=4 mice for BL+B. *p<0.05, ** p<0.01 for BL+B compared to BL+RK (t-tests).

FIGS. 5A-5B are graphs quantifying the number of cells detected in mouse bronchoalveolar lavage fluid. FIG. 5A quantifies the total number of detected cells. FIG. 5B quantifies lymphocytes detected by Wright-Giemsa staining. Points indicate individual mouse values for saline with buffer (S+B), saline with RK33 (S+RK), bleomycin with buffer (BL+B), and bleomycin with RK33 (BL+RK), respectively. Values are mean SEM, n=5 mice for S+B, S+RK, and BL+RK, n=4 mice for BL+B. ns=not significant, *p<0.05, **p<0.01, ***p<0.001 (1-way ANOVA, Dunnett's test).

FIGS. 6A-6B: FIG. 6A are images of PicroSirius-red stained mouse lung sections. The top-left image shows a lung section from a mouse given saline with buffer (S+B), the top-right image shows a lung section from a mouse given saline with RK33 (S+RK), the bottom-left image shows a lung section from a mouse given bleomycin with buffer (BL+B), the bottom-right image shows a lung section from a mouse given bleomycin with RK33 (BL+RK). Bar is 100 μm. FIG. 6B quantifies the observed collagen by PicroSirius-red staining. Points indicate individual mouse values for saline with buffer (S+B), saline with RK33 (S+RK), bleomycin with buffer (BL+B), and bleomycin with RK33 (BL+RK), respectively. Values are mean SEM, n=5 mice for S+B, S+RK, and BL+RK, n=4 mice for BL+B. ns=not significant, **p<0.01, ***p<0.001 (1-way ANOVA, Dunnett's test).

FIG. 7 shows quantification of collagen in mouse lungs with a hydroxyproline assay. Points indicate individual mouse values for saline with buffer (S+B), saline with RK33 (S+RK), bleomycin with buffer (BL+B), and bleomycin with RK33 (BL+RK), respectively. Values are mean±SEM, n=5 mice for S+B, S+RK, and BL+RK, n=4 mice for BL+B. ns=not significant, *p<0.05, ***p<0.001 (1-way ANOVA, Dunnett's test).

### DETAILED DISCLOSURE OF THE INVENTION

The present disclosure relates to compounds and compositions comprising one or more inhibitor of DDX3 and methods for suppressing the pro-fibrosis effects of factors such as TGF-β1 or IL-6 by administration of one or more DDX3 inhibitor to a subject or in an in vitro setting. Non-limiting examples of DDX3 inhibitors are found, for example, in Yedavalli et al., J. Med. Chem., 2008, 51:5043-51; Maga et al., J. Med. Chem., 2008, 51:6635-38; Maga et al., Chem. Med. Chem., 2011:6:1371-89; Radi et al., Bioorg. Med. Chem. Lett., 2012, 22:2094-98; Samal et al., Sci. Rep., 2015, 5:9982; Bol et al., EMBO Mol. Med., 2015, 7:648-69; Fazi et al., J. Chem. Inf. Model., 2015, 55:2443-54; Brai et al., Proc. Natl. Acad. Sci. USA, 2016, 113:5388-93; Garbelli et al., Curr. Med. Chem., 2011, 18:3015-27; Gherardini et al., Cancers, 2021, 13:4830; Brai et al., WO 2017/162834; Rampogu et al., Front. Oncol., 2021, 11:712824; Fu et al., Cell Death Dis., 2019, 10:593; Rampogu et al., Biomolecules, 2020, 10:857; Chen et al., Cell. Chem. Biol., 2020, 28:475-86; U.S. Patent Application Publication 2018/0016243A1; U.S. Pat. No. 10,941,121; WO 2010/039187; and WO 2015/200779, each of which is hereby incorporated by reference in its entirety, particularly with respect to the DDX3 inhibitors disclosed therein. Tables 1-16 and 17A-17B also provide non-limiting examples of DDX3 inhibitors disclosed in these documents.

In the context of the present invention, the term “suppressing the pro-fibrosis effects of factors such as TGF-β1 and/or IL-6” refers to one or more of inhibiting and/or decreasing the ability of TGF-β1 to increase levels of NEU3, inhibiting and/or decreasing the ability of IL-6 to increase levels of NEU3, inhibiting and/or decreasing the ability of TGF-β1 to increase levels of proteins in addition to NEU3, or inhibiting and/or decreasing the ability of IL-6 to increase levels of proteins in addition to NEU3. In some embodiments, fibrosis in a subject can be inhibited or reduced. This process may also be referred to as “a method of suppressing fibrosis . . . ” or “a method of treating fibrosis . . . ” within this disclosure.

The terms “treat”, “treating”, “inhibiting”, “inhibit”, “suppressing”, “suppress”, “decrease” and “decreasing” are used, in the context of this disclosure, to refer to the reduction, improvement, stabilization and/or elimination of a symptom of a disease, or slowing the progression of a disease associated with the pro-fibrosis effects of TGF-β1 and/or IL-6 by decreasing levels of NEU3. Such diseases are disclosed below and are treated by way of the administration of one or more inhibitor of DDX3 alone or in combination with one or more of serum amyloid P (SAP) or fragments thereof, IL-12, Laminin-1, IgG aggregates, cross-linked IgG, cofactors of any of the above, and any combination thereof.

The term “therapeutically effective amount” is intended to constitute an amount of one or more DDX3 inhibitor that treats or suppresses fibrosis in a subject. In other words, one or more inhibitor of DDX3 is administered in an amount that the therapeutic product of the invention (the “inhibitor”) administered to a subject that is sufficient to constitute a treatment of the disease and cause a reduction, improvement, stabilization and/or elimination of a symptom of a disease, or slowing the progression of a disease associated with the pro-fibrosis effects of TGF-β1 and/or IL-6 by decreasing levels of NEU3. Such amounts could range from about 0.1 to about 100 mg/kg of a subject to be treated.

The terms “about” or “approximately” mean a range of ±0-20%, ±0 to 10%, or up to ±1% of a given value. In the context of compositions containing amounts of ingredients where the terms “about” or “approximately” are used, these compositions contain the stated amount of the ingredient with a variation (error range) of 0-10% around the value (X±10%). With respect to periods of time (days, weeks, months), the term is intended to include a period of 6 hours for days, ±1 day for weeks, and +7 days for months.

The term “and/or” is used, herein, as a function word to indicate that two words or expressions are to be taken together or individually. Thus, in the case of “suppressing the pro-fibrosis effects of factors such as TGF-β1 and/or IL-6”, the disclosed methods suppress: a) the pro-fibrosis effects of TGF-β1 alone; b) the pro-fibrosis effects of IL-6 alone; or c) the pro-fibrosis effects caused by the combined effects of TGF-β1 and IL-6.

As used herein, the singular forms “a”, “an” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, include the phrases “consisting essentially of”, “consists essentially of”, “consisting”, and “consists” can be used interchangeably. The phrases “consisting essentially of” or “consists essentially of” indicate that the claim encompasses embodiments containing the specified materials or steps and those that do not materially affect the basic and novel characteristic(s) of the claim.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. When ranges are used herein (such as for dose ranges), specific embodiments of different combinations and subcombinations of these dose ranges (e.g., subranges within the disclosed ranges) are intended to be explicitly included.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art.

In some embodiments, fibrosis can be suppressed by a combination treatment using the compounds and compositions comprising one or more inhibitor of DDX3 in combination with one or more of serum amyloid P (SAP) or fragments thereof, IL-12, Laminin-1, IgG aggregates, cross-linked IgG, cofactors of any of the above, and any combination thereof. In particular embodiments, one or more inhibitors of DDX 3 are administered in combination with SAP (full length SAP) and/or one or more fragment of SAP, said fragments being, for example, KERVGEYSLYIGRHKVTSKVIEKFP (SEQ ID NO: 1), ILSAYQGTPLPA (SEQ ID NO: 2), IRGYVIIKPLV (SEQ ID NO: 3), or combinations thereof. In the context of this invention, where fragments of SAP are claimed with the use of “comprising” language, such language specifically excludes full length SAP. The use of serum amyloid P (SAP), IL-12, Laminin-1, IgG aggregates, cross-linked IgG, cofactors of any of the above, and any combination thereof in the suppression of fibrocytes has been disclosed, for example, in U.S. Pat. No. 8,187,608, the disclosure of which is hereby incorporated by reference in its entirety.

As used herein, the term “in combination” refers to a combination of active agents, in this case one or more DDX3 inhibitor and one or more of serum amyloid P (SAP) or fragments thereof, IL-12, Laminin-1, IgG aggregates, cross-linked IgG, cofactors of any of the above, and any combination thereof, which may each be formulated separately or as a combined formulation (composition) simultaneous, separate or sequential administration, or a mixture of these modes of administration.

As used herein, the term “simultaneous” refers to a combination according to the invention in which the active agents of the combination are used or administered simultaneously, i.e. at the same time.

As used herein, the term “sequential” refers to a combination according to the invention in which the active agents of the combination are used or administered sequentially, i.e. one after the other. Preferably, when the administration is sequential, all of the active principles are administered within an interval of not more than about 1 hour, preferably not more than about 10 minutes, and more preferably not more than about 1 minute.

As used herein, the term “separate” refers to a combination according to the invention in which the active agents of the combination are used or administered at separate times in the day. Preferably, when the administration is separate, the active agents are administered at intervals from about 1 hour to about 15 hours, preferably from about 1 hour to about 8 hours, and more preferably from about 1 hour to about 5 hours.

The active agents disclosed herein (e.g., one or more DDX3 inhibitor and/or serum amyloid P (SAP) or fragments thereof, IL-12, Laminin-1, IgG aggregates, cross-linked IgG, cofactors of any of the above, and any combination thereof) can be formulated into pharmaceutically acceptable compositions. Such compositions comprise one or more of the disclosed active agents in combination with a pharmaceutically acceptable carrier such as a phosphate buffered saline, a bicarbonate solution, or formulated with a carrier such as starch into a pill to produce a pharmaceutical composition. The carrier must be “acceptable” in the sense that it is compatible with the active ingredient (agent) of the composition, and preferably capable of stabilizing the active agent and not deleterious to the subject to be treated. The carrier is selected on the basis of the mode and route of administration and standard pharmaceutical practice. Suitable pharmaceutical carriers and diluents, as well as pharmaceutical necessities for their use, are described in Remington's Pharmaceutical Sciences.

“Subject” refers to an animal, such as a mammal, for example a human. The methods described herein can be useful in both pre-clinical human therapeutics and veterinary applications. In some embodiments, the subject is a mammal (such as an animal model of disease, e.g., a mouse or rat), and in some embodiments, the subject is human. The terms “subject” “mammal” and “patient” can be used interchangeably.

The compounds and compositions disclosed herein can be administered by any acceptable route for the treatment of a fibrosing disease or disorder caused by fibrosis in a subject. Non-limiting examples include administering the compounds and/or compositions disclosed herein orally, by injection, by subcutaneous injection, by intravenous infusion, topically, or by inhalation. In some embodiments, the compounds and/or compositions are administered into a target location (a tissue and/or organ) undergoing fibrosis or into a site of fibrosis.

The target location may be located in vitro or in vivo. Specifically, the target location may be located in a mammal, such as a human patient, and includes tissues and/or organs undergoing fibrosis. In vivo, the target location may include an entire organism or a portion thereof and the composition may be administered systemically or it may be confined to a particular area, such as an organ or tissue where the compositions are directly administered to sites of fibrosis within an organ and/or tissue. The compositions may be supplied directly in (or to) a target location or administered systemically.

Suppressing the pro-fibrosis effects of factors such as TGF-β1 and/or IL-6 may alleviate symptoms of numerous fibrosing diseases or other disorders caused by fibrosis, such as those identified in the section entitled “Fibrosing Targets” below. In a specific embodiment, administration of one or more inhibitor of DDX3 and, optionally, SAP and/or fragments thereof, IL-12, Laminin-1, IgG aggregates, cross-linked IgG, cofactors of any of the above, and/or any combination thereof may be used to treat the effects of unwanted fibrosis caused by pro-fibrotic factors such as TGF-β1 and/or IL-6. For example, it may be used to treat fibrosis in the liver, kidney, lung, heart and pericardium, eye, skin, mouth, pancreas, gastrointestinal tract, brain, breast, bone marrow, bone, genitourinary, a tumor, or a wound.

Fibrosis can be suppressed or treated by administering compounds and compositions comprising one or more inhibitor of DDX3 alone or in combination with one or more of serum amyloid P (SAP) or fragments thereof, IL-12, Laminin-1, IgG aggregates, cross-linked IgG, cofactors of any of the above, and any combination thereof over a period of time (e.g., at discrete time points separated by days, week, or months). In some embodiments, the compounds and compositions comprising one or more inhibitor of DDX3 alone or in combination with one or more of serum amyloid P (SAP) or fragments thereof, IL-12, Laminin-1, IgG aggregates, cross-linked IgG, cofactors of any of the above, and any combination thereof can be administered a certain number of times per week or month (for example, 1, 2, 3, 4, 5, 6, or 7 times per week or between 1 day and x days per month, where x is 28, 29, 30, or 31 days depending on the month or year (if a leap year)). “A week” refers to a period of time of about 5, about 6 or about 7 days. “A month” refers to a period of time of about 28, about 29, about 30 or about 31 days.

In yet other embodiments, the administration of compounds and compositions comprising one or more inhibitor of DDX3 alone or in combination with one or more of serum amyloid P (SAP) or fragments thereof, IL-12, Laminin-1, IgG aggregates, cross-linked IgG, cofactors of any of the above, and any combination thereof can be separated by a period of time. The length of time between the different points in time where the treatment is administered can range between 1 to 30 days, between about 1 and about 52 weeks, or can be 1, 2, 3, 4, 5, 6, 7, 8, 9, 10, 11, or up to 12 months between each doses of at least one DDX3 inhibitor and/or one or more of serum amyloid P (SAP) or fragments thereof, IL-12, Laminin-1, IgG aggregates, cross-linked IgG, cofactors of any of the above, and any combination thereof.

As discussed above, therapeutically effective amounts of one or more DDX3 inhibitor are administered to a subject in need of treatment of fibrosis of for the suppression of fibrosis in a subject. Thus, one or more DDX3 inhibitor can be administered in an amount that ranges from about 0.1 mg/kg to about 100 mg/kg of a subject to be treated; about 1 mg/kg to about 75 mg/kg of a subject to be treated; about 1 mg/kg to about 50 mg/kg of a subject to be treated; about 10 mg/kg to about 100 mg/kg of a subject to be treated; about 10 mg/kg to about 75 mg/kg of a subject to be treated; about 10 mg/kg to about 50 mg/kg of a subject to be treated; about 1 mg/kg to about 75 mg/kg of a subject to be treated; about 10 mg/kg to about 30 mg/kg of a subject to be treated; about 15 mg/kg to about 30 mg/kg of a subject to be treated; about 15 mg/kg to about 25 mg/kg of a subject to be treated; or in an amount of about 20 mg/kg of a subject to be treated.

### Fibrosing Targets

The disclosed compositions may also be used to treat diseases or conditions resulting from inappropriately high levels of NEU3, for example a fibrosing disease. For example, the disclosed compositions can be used to treat a fibrosis occurring in the liver, kidney, lung, heart and pericardium, eye, skin, mouth, pancreas, gastrointestinal tract, brain, breast, bone marrow, bone, genitourinary, a tumor, or a wound.

Non-limiting examples of such fibrotic diseases or conditions include: rheumatoid arthritis, lupus, pathogenic fibrosis, fibrosing disease, fibrotic lesions such as those formed after Schistosoma japonicum infection, radiation damage, autoimmune diseases, Lyme disease, chemotherapy induced fibrosis, HIV or infection-induced focal sclerosis, failed back syndrome due to spinal surgery scarring, abdominal adhesions, post-surgical scarring, fibrocystic formations, fibrosis after spinal injury, surgery-induced fibrosis, mucosal fibrosis, peritoneal fibrosis caused by dialysis, and Adalimumab-associated pulmonary fibrosis.

In some embodiments, the compositions disclosed herein can be used to treat or prevent fibrosis of the liver that results from conditions including but not limited to alcohol, drug, and/or chemically induced cirrhosis, ischemia-reperfusion injury after hepatic transplant, necrotizing hepatitis, hepatitis B, hepatitis C, primary biliary cirrhosis, and primary sclerosing cholangitis.

In other embodiments, the compositions disclosed herein can be used to treat fibrosis arising in the kidney, including but not limited to: proliferative and sclerosing glomerulonephritis, nephrogenic fibrosing dermopathy, diabetic nephropathy, renal tubulointerstitial fibrosis, and focal segmental glomerulosclerosis.

Yet other embodiments contemplate treating fibrosis that is associated with the lungs. Non-limiting examples of such diseases include: pulmonary interstitial fibrosis, sarcoidosis, pulmonary fibrosis, idiopathic pulmonary fibrosis, asthma, chronic obstructive pulmonary disease, diffuse alveolar damage disease, pulmonary hypertension, neonatal bronchopulmonary dysplasia, chronic asthma, and emphysema. There are several sub-names or synonyms for pulmonary fibrosis including, but not limited to, cryptogenic fibrosing alveolitis, diffuse interstitial fibrosis, idiopathic interstitial pneumonitis, Hamman-Rich syndrome, silicosis, asbestosis, berylliosis, coal worker's pneumoconiosis, black lung disease, coal miner's disease, miner's asthma, anthracosis, and anthracosilicosis.

Fibrosing disease also occurs for the heart and/or pericardium. Thus, various embodiments contemplate the treatment of myocardial fibrosis, atherosclerosis, coronary artery restenosis, congestive cardiomyopathy, heart failure, and other post-ischemic conditions that comprise the administration of compositions disclosed herein.

The compositions disclosed herein can also be used to treat fibrosing diseases of the eye, such as exophthalmos of Grave's disease, proliferative vitreoretinopathy, anterior capsule cataract, corneal fibrosis, corneal scarring due to surgery, trabeculectomy-induced fibrosis, progressive subretinal fibrosis, multifocal granulomatous chorioretinitis, and other eye fibrosis.

Likewise, fibrosis diseases affecting the skin can also be treated by the administration of compositions disclosed herein to the skin of a subject. Non-limiting examples of fibrotic diseases of the skin include: Dupuytren's contracture, scleroderma, keloid scarring, psoriasis, hypertrophic scarring due to burns, atherosclerosis, restenosis, and psuedoscleroderma caused by spinal cord injury.

The disclosed compositions are also suitable for the treatment of fibrotic diseases of the mouth and/or esophagus; they may treat or prevent fibrosis resulting from conditions including but not limited to periodontal disease scarring, gingival hypertrophy secondary to drugs, and congenital esophageal stenosis.

Compositions disclosed herein are also suitable for the treatment of fibrotic diseases affecting the pancreas. Non-limiting examples of such diseases include: pancreatic fibrosis, stromal remodeling pancreatitis, and stromal fibrosis.

Compositions disclosed herein are also suitable for the treatment of fibrotic diseases affecting the gastrointestinal tract. Non-limiting examples of such diseases include: collagenous colitis, villous atrophy, crypt hyperplasia, polyp formation, fibrosis of Crohn's disease, and healing gastric ulcer.

Fibrotic conditions affecting the brain can also be treated with compositions disclosed herein. Such conditions include, and are not limited to: glial scar tissue.

Fibrotic conditions of the breast can also be treated using the disclosed compositions. For example, fibrocystic disease of the breast and desmoplastic reaction to breast cancer can be treated with the disclosed compositions.

The disclosed compositions can also be used to treat fibrosis arising in the bone marrow that can result from conditions including, but not limited to, fibrosis in myelodysplasia, myelofibrosis, and neoplastic diseases. Similarly, fibrotic diseases associated with the bone can be treated using the disclosed compositions. For example, the disclosed compositions can be used to treat rheumatoid pannus formation.

Fibrotic diseases/conditions of the genitourinary system can also be treated with the disclosed compositions. Such diseases/conditions include, but are not limited to, endometriosis, uterine fibroids, ovarian fibroids, and Peyronie's disease.

Additionally, the disclosed compositions can be used to treat fibrosis caused by radiation including radiation-induced damage used to treat various forms of cancer, such as head and neck cancer, ovarian cancer, prostate cancer, lung cancer, gastrointestinal cancer, colon cancer, and breast cancer.

The following examples are included to demonstrate specific embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

TABLE 1
DDX3 inhibitors from Bol et al., 2015

| Commercial Name | Compound name |
|---|---|
| RK-33 | (diimidazo[4,5-d:4′,5′-f]-[1,3]diazepine) |

TABLE 2
DDX3 inhibitors from Brai et al., 2016

| Reference Name | Compound name |
|---|---|
| Compound 1 | 1-(o-Tolyl)-3-[3-(o-tolylcarbamoylamino)phenyl]urea |
| Compound 2 | 1-(3-Nitrophenyl)-3-o-tolylurea |
| Compound 3 | 1-(3-Aminophenyl)-3-o-tolylurea |
| Compound 9 | 1-(Naphthalen-1-yl)-(3-nitrophenyl)urea |
| Compound 10 | 1-Cyclohexyl-3-(3-nitrophenyl)urea |
| Compound 11 | Ethyl 3-(3-o-tolylureido)benzoate |
| Compound 12 | 3-(3-o-Tolylureido)benzoic acid |
| Compound 16a | 1-(4-(4-Phenyl-1H-1,2,3-triazol-1-yl)phenyl)-3-o-tolylurea |
| Compound 16b | 1-(4-(4-Tert-butyl-1H-1,2,3-triazol-1-yl)phenyl)-3-o-tolylurea |
| Compound 16c | 1-(4-(4-(Hydroxymethyl)-1H-1,2,3-triazol-1-yl)phenyl)-3-o-tolylurea |
| Compound 16d | 1-(4-(4-Methyl-1H-1,2,3-triazol-1-yl)phenyl)-3-o-tolylurea |
| Compound 16e | 1-(4-(4-((Benzyl(methyl)amino)methyl)-1H-1,2,3-triazol-1-yl) |
| | phenyl)-3-(o-tolyl)urea |
| Compound 16f | 1-(4-(4-Methyl-1H-1,2,3-triazol-1-yl)phenyl)-3-o-tolylurea |
| Compound 16g | 1-(4-(4-Ethyl-1H-1,2,3-triazol-1-yl)phenyl)-3-o-tolylurea |

TABLE 3
DDX3 inhibitors from U.S. Pat. No. 11,000,512B2 (Brai et al.)

| Reference Name | Compound name |
|---|---|
| Compound 8a | 1-(4-(4-phenyl-1H-1,2,3-triazol-1-yl)phenyl)-3-o-tolylurea |
| Compound 20a | 1-(4-(4-butyl-1H-1,2,3-triazol-1-yl)phenyl)-3-o-tolylurea |
| Compound 8b | 1-(4-(4-tert-butyl-1H-1,2,3-triazol-1-yl)phenyl)-3-o-tolylurea |
| Compound 8c | 1-(4-(4-methanamine,N-[(phenyl)methyl]-N-methyl-1H-1,2,3-triazol- |
| | 1yl)phenyl)-3-o-tolylurea |
| Compound 8g | 1-(4-(4-ethyl-1H-1,2,3-triazol-1-yl)phenyl)-3-o-tolylurea |
| Compound 20b | 1-(4-(4-isopentyl-1H-1,2,3-triazol-1-yl)phenyl)-3-o-tolylurea |
| Compound 20e | 1-(4-(4-(ethoxymethyl)-1H-1,2,3-triazol-1-yl)phenyl)-3-(o-tolyl)urea |
| Compound 20f | 1-(4-(4-(2-methoxyethyl)-1H-1,2,3-triazol-1-yl)phenyl)-3-o-tolylurea |
| Compound 22a | 1-(4-(4-butyl-1H-1,2,3-triazol-1-yl)phenyl)-3-(2- |
| | (trifluoromethyl)phenyl)urea |
| Compound 22b | 1-(4-(4-isopentyl-1H-1,2,3-triazol-1-yl)phenyl)-3-(2- |
| | (trifluoromethyl)phenyl)urea |
| Compound 35a | 1-(4-(4-(hydroxymethyl)-1H-1,2,3-triazol-1-yl)phenyl)-3-o-tolylurea |
| Compound 35b | 1-(4-(4-(hydroxyethyl)-1H-1,2,3-triazol-1-yl)phenyl)-3-o-tolylurea |
| Compound 35h | 4-(4-(3-(4-methylpiperazin-1-yl)propyl)-1H-1,2,3-triazol-1- |
| | yl)benzenamine |
| Compound 35i | 1-(4-(4-(dimethylaminopropyl)-1H-1,2,3-triazol-1-yl)phenyl)-3-o- |
| | tolylurea |
| Compound 36 | diethyl (2-(1-(4-(3-(o-tolyl)ureido)phenyl)-1H-1,2,3-triazol-4-yl)ethyl) |
| | phosphate |
| Compound 37 | 2-(1-(4-(3-(o-tolyl)ureido)phenyl)-1H-1,2,3-triazol-4-yl)ethyl 3- |
| | methylbutanoate |
| Compound 38 | 2-(1-(4-(3-(2-(trifluoromethyl)phenyl)ureido)phenyl)-1H-1,2,3-triazol- |
| | 4-yl)ethyl 3-methylbutanoate |
| Compound 39 | 2-(1-(4-(3-(2-(trifluoromethyl)phenyl)ureido)phenyl)-1H-1,2,3-triazol- |
| | 4-yl)ethyl 3-(benzo[d][1,3]dioxol-5-yl)acrylate |
| Compound 42c | 1-(4-(4-(1-fluoro-2-methylpentyl)-1H-1,2,3-triazol-1-yl)phenyl)-3-(2- |
| | (trifluoromethyl) phenyl) urea |
| Compound 20c | 1-(4-(4-(perfluorobutyl)-1H-1,2,3-triazol-1-yl)phenyl)-3-(o-tolyl)urea |
| Compound 81 | 1-(4-(4-butyl-1H-1,2,3-triazol-1-yl)-3-fluorophenyl)-3-(2- |
| | (trifluoromethyl) phenyl)urea |
| Compound 49 | 1-(4-(2-butyl-2H-tetrazol-5-yl)phenyl)-3-(o-tolyl)urea |
| Compound 50 | 1-(4-(2-(ethoxymethyl)-2H-tetrazol-5-yl)phenyl)-3-(o-tolyl)urea |
| Compound 51 | 1-(4-(2-butyl-2H-tetrazol-5-yl)phenyl)-3-(2- |
| | (trifluoromethyl)phenyl)urea |
| Compound 52 | 1-(4-(2-butyl-2H-tetrazol-5-yl)phenyl)-3-(4-methylpyridin-3-yl)urea |
| Compound 55a | 1-(2-fluorophenyl)-3-(4-(4-isopentyl-1H-1,2,3-triazol-1-yl)phenyl)urea |
| Compound 55b | 1-(3-fluorophenyl)-3-(4-(4-isopentyl-1H-1,2,3-triazol-1-yl)phenyl)urea |
| Compound 55e | 1-(4-(4-(ethoxymethyl)-1H-1,2,3-triazol-1-yl)phenyl)-3-(5-isopropyl-2- |
| | methyl phenyl) urea |
| Compound 55f | 1-(4-(4-isopentyl-1H-1,2,3-triazol-1-yl)phenyl)-3-(isoquinolin-5- |
| | yl)urea |
| Compound 55g | 1-(1-chloro-3-methylisoquinolin-4-yl)-3-(4-(4-isopentyl-1H-1,2,3- |
| | triazol-1-yl)phenyl)urea |
| Compound 15a | 1-(4-(4-butyl-1H-1,2,3-triazol-1-yl)phenyl)-3-(4-methylpyridin-3- |
| | yl)urea |
| Compound 15b | 1-(4-(4-isopentyl-1H-1,2,3-triazol-1-yl)phenyl)-3-(4-methylpyridin-3- |
| | yl)urea |
| Compound 58a | N-(2-hydroxy)-3-nitro-phenylbenzenesulfonamide |
| Compound 64a | 3-(4-butyl-1H-1,2,3-triazol-1-yl)-N-(2- |
| | hydroxyphenyl)benzenesulfonamide |
| Compound 64b | 3-(4-butyl-1H-1,2,3-triazol-1-yl)-N-(4- |
| | methoxyphenyl)benzenesulfonamide |
| Compound 64d | 3-(4-butyl-1H-1,2,3-triazol-1-yl)-N-(2- |
| | (trifluoromethyl)phenyl)benzenesulfonamide |
| Compound 66d | 3-(4-(ethoxymethyl)-1H-1,2,3-triazol-1-yl)-N-(2- |
| | (trifluoromethyl)phenyl) benzenesulfonamide |
| Compound 64e | 3-(4-butyl-1H-1,2,3-triazol-1-yl)-N-(isoquinolin-6- |
| | yl)benzenesulfonamide |
| Compound 68e | 3-(4-(ethoxymethyl)-1H-1,2,3-triazol-1-yl)-N-(isoquinolin-6- |
| | yl)benzenesulfonamide |
| Compound 65a | 4-(4-butyl-1H-1,2,3-triazol-1-yl)-N-(2- |
| | hydroxyphenyl)benzenesulfonamide |
| Compound 134 | 3-(5-butyl-1,2,4-oxadiazol-3-yl)-N-(2- |
| | (trifluoromethyl)phenyl)benzenesulfonamide |
| Compound 112 | 1-(4-(5-butyl-1,3,4-oxadiazol-2-yl)phenyl)-3-(2- |
| | (trifluoromethyl)phenyl)urea |
| Compound 106 | 1-(4-(5-butyl-1,2,4-oxadiazol-3-yl)phenyl)-3-(2- |
| | (trifluoromethyl)phenyl)urea |
| Compound 8e | 1-(4-(4-butyl-1H-1,2,3-triazol-1-yl)-2-methoxyphenyl)-3-(2- |
| | (trifluoromethyl)phenyl)urea |
| Compound 80 | 1-(4-(4-isopentyl-1H-1,2,3-triazol-1-yl)-3-methoxyphenyl)-3-(2- |
| | (trifluoromethyl) phenyl)urea |
| Compound 86 | 1-(6-(4-isopentyl-1H-1,2,3-triazol-1-yl)pyridin-3-yl)-3-(2- |
| | (trifluoromethyl) phenyl)urea |
| Compound 55h | 1-(4-(4-butyl-1H-1,2,3-triazol-1-yl)phenyl)-3-(5-(cyclopentyloxy)-2- |
| | methylphenyl)urea |
| Compound 124 | 1-(4-(5-butyl-1,3,4-thiadiazol-2-yl)phenyl)-3-(2- |
| | (trifluoromethyl)phenyl)urea |
| Compound 119 | 1-(4-(5-butyl-4H-1,2,4-triazol-3-yl)phenyl)-3-(2- |
| | (trifluoromethyl)phenyl)urea |

TABLE 4
DDX3 inhibitors from Fazi et al., 2015

| Reference Name | Commercial Name | Compound name |
|---|---|---|
| Compound 3 | Asinex BAS | (2-(2-Benzoylhydrazinecarbonyl)benzoic |
| | 00569365 | acid) |
| Compound 4 | Asinex BAS | (2-Methoxy-6-(quinolin-8-ylaminomethyl)- |
| | 05346780 | phenol) |
| Compound 5 | Asinex BAS | 3-Methyl-benzoic acid N′-(2-nitro-phenyl)- |
| | 00605530 | hydrazide |
| Compound 6 | Asinex BAS | (N-(3-hydroxyphenyl)-2-(4- |
| | 00298315 | methoxyphenyl)-1,3-dioxo-2,3-dihydro-1H- |
| | | isoindole-5-carboxamide) |
| Compound 7 | Asinex BAS | (Benzoic acid N′-(2-nitro-4-trifluoromethyl- |
| | 00313098 | phenyl)-hydrazide) |
| Compound 8 | Asinex BAS | 4,5-Dimethyl-2-(3-Nitro-Benzoylamino)- |
| | 00541498 | Thiophene-3-Carboxylic Acid Amide |
| Compound 9 | Asinex BAS | N-{4-[3-(1H-Benzoimidazol-2-Y1)- |
| | 00844166 | Phenylsulfamoyl]-Phenyl}-Acetamide |
| Compound 10 | Asinex BAS | 3-(2-Amino-Phenylamino)-5,5-Dimethyl- |
| | 03161011 | Cyclohex-2-Enone |
| Compound 11 | Asinex BAS | 1-Benzo[1,2,3]Thiadiazol-5-Yl-3-Benzyl- |
| | 04323716 | Urea |
| Compound 12 | Chembridge | N-[3-(hydrazinocarbonyl)phenyl]-2- |
| | 5261271 | methylbenzenesulfonamide |
| Compound 13 | Chembridge | (5-[(3,4-Dimethylphenyl)sulfamoyl]-2- |
| | 7779729 | hydroxybenzamide) |
| Compound 14 | Chembridge | N-(2-hydroxyphenyl)-3- |
| | 5227931 | nitrobenzenesulfonamide |
| Compound 15 | Chembridge | N-(2-Hydroxyphenyl)-3-nitrobenzamide |
| | 5181912 | |
| Compound 16 | Chembridge | (1-(3-Nitrophenyl)-3-(2,4,4- |
| | 7989453 | trimethylpentan-2-yl)urea) |
| Compound 17 | IBS-STOCK6S- | N-(2-(dimethylamino)ethyl)-2-(4-oxo-3,4- |
| | 20585 | dihydrophthalazin-1-yl)acetamide |
| | | hydrochloride |
| Compound 18 | IBS-STOCK6S- | N-(2-(4-((1-methyl-1H-benzo[d]imidazol-2- |
| | 72571 | yl)methyl)piperazin-1-yl)phenyl)furan-2- |
| | | carboxamide |
| Compound 19 | IBS-STOCK6S- | N-(2-(4-((1-methyl-1H-benzo[d]imidazol-2- |
| | 14656 | yl)methyl)piperazin-1-yl)phenyl)furan-2- |
| | | carboxamide |
| Compound 20 | SPECS AN- | N′-(5,5-dimethyl-3-oxo-1-cyclohexen-1-yl)- |
| | 329/13193015 | 2-methyl-3-furohydrazide |
| Compound 21 | SPECS AG- | 4-(Benzenesulfonamido)-3- |
| | 690/36490010 | hydroxynaphthalene-1-sulfonic acid |
| Compound 22 | SPECS AQ- | 3-(1H-benzimidazol-2-ylamino)benzoic |
| | 086/41476304 | acid |
| Compound 23 | SPECS AP- | N~1~,N~5~-bis(2-methoxyphenyl)-1,5- |
| | 263/40222373 | naphthalenedisulfonamide |
| Compound 24 | SPECS AQ- | 5-(1H-benzimidazol-2-ylamino)-2- |
| | 086/43457605 | hydroxybenzoic acid |
| Compound 25 | SPECS AG- | N′-(5,5-dimethyl-3-oxo-1-cyclohexen-1-yl) |
| | 205/08449028 | isonicotinohydrazide |
| Compound 26 | SPECS AK- | 6-{[2-(2-methyl-3-furoyl)hydrazino] |
| | 968/41026362 | carbonyl}-3-cyclohexene-1-carboxylic acid |
| Compound 27 | SPECS AN- | N′-butyryl-2-methyl-3-furohydrazide |
| | 329/40719029 | |

TABLE 5
DDX3 inhibitors from Garbelli et al., 2011

| | | | Formula (I) |
|---|---|---|---|
| 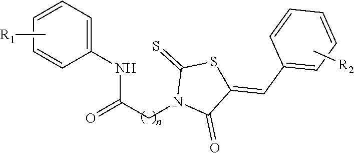 | | | |
| Compound | R1 | R2 | n |
| Fe02 | 3-OH | 2-OCH₃ | 2 |
| Fe10 | 3-OH | 3-Br | 2 |
| Fe14 | 3-OH | 3-Br | 3 |
| Fe15 | 2-OH | 3-Br | 2 |
| Fe16 | 4-OH | 3-Br | 2 |
| Fe20 | 2-OH | 3-Br | 3 |
| Fe21 | 2-COOH | 3-Br | 2 |
| Fe22 | 3-OH | 3-Br | 2 |
| | | | Formula (II) |
| 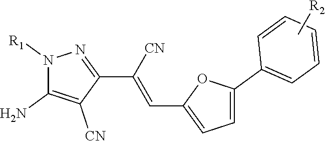 | | | |
| Compound | R1 | R2 | N |
| Fe06 | Phenyl | 2-NO₂ | — |
| Fe13 | H | 3-NO₂ | — |
| Fe18 | Phenyl | 3-Cl | — |

TABLE 6
DDX3 inhibitors from Gherardini et al., 2021

| Commercial Name | Compound name |
|---|---|
| FHP01 | 1-(4-(4-butyl-1H-1,2,3-triazol-1-yl)phenyl)-3-(2- |
| | (trifluoromethyl)phenyl)urea |

TABLE 7
DDX3 inhibitors from Chen et al., 2020

| Commercial | |
|---|---|
| Name | Compound name |
| Rocaglamide A | (1R,2R,3aR,8bS)-1,8b-dihydroxy-6,8-dimethoxy-3a-(4- |
| | methoxyphenyl)-N,N-dimethyl-3-phenyl-2,3-dihydro- |
| | 1H-cyclopenta[b][1]benzofuran-2-carboxamide |

TABLE 8
DDX3 inhibitors from Rampogu et al., 2021

| Commercial/Reference Name | Compound name |
|---|---|
| Butein | 2′,3,4,4′-Tetrahydroxychalcone |
| 3a | (E)-3-(5-bromothiophen-2-yl)-1-(2,4- |
| | dihydroxyphenyl)prop-2-en-1-one |
| 3b | (E)-3-(5-bromofuran-2-yl)-1-(2,4- |
| | dihydroxyphenyl)prop-2-en-1-one |
| 3c | (E)-1-(2,4-dihydroxyphenyl)-3-(m-tolyl) |
| | prop-2-en-1-one |

TABLE 9
DDX3 inhibitors from Fu et al., 2019

| Commercial Name | Compound name |
|---|---|
| avenanthramide A | 5-Hydroxy-2-{[(2E)-3-(4-hydroxyphenyl)-1-oxo-2- |
| | propenyl]amino}benzoic acid, N-(4′-Hydroxy-(E)-cinnamoyl)-5- |
| | hydroxyanthranilic acid |

TABLE 10
DDX3 inhibitors from Rampogu et al., 2020

| Commercial Name | Compound name |
|---|---|
| Curcumin | Diferuloylmethane |
| exemestane | 6-Methyleneandrosta-1,4-diene-3,17-dione |

TABLE 11
DDX3 inhibitors from Radi et al., 2012

| Reference Name | Compound name |
|---|---|
| Compound 1 | 1-(o-Tolyl)-3-[3-(o-tolylcarbamoylamino)phenyl]urea |
| Compound 2 | N-[4-(2,3-dihydro-1H-benzimidazol-2-yl)phenyl]-2,3- |
| | dihydrobenzofuran-2-carboxamide |
| Compound 3 | 3-(3,7-diphenyl-4H-diazepin-5-yl)-2-methyl-1H-indole |
| Compound 6 | 1,3-Bis(3-nitrophenyl)urea |
| Compound 7 | 1-(3-Nitrophenyl)-3-o-tolylurea |
| Compound 8 | 1-(3-Aminophenyl)-3-o-tolylurea |
| Compound 9 | 1,3-Bis(3-aminophenyl)urea |

TABLE 12
DDX3 inhibitors from JP6749344B2 (U.S. application
Ser. No. 15/550, 155; 2018/0016243A1)

| Reference | |
|---|---|
| Name | Compound name |
| 8f | 1-(4-(4-methyl-1H-1,2,3-triazol-1-yl)phenyl)-3-o-tolylurea |
| 22a | 1-(4-(4-butyl-1H-1,2,3-triazol-1-yl)phenyl)-3-(2-(trifluoromethyl)phenyl)urea |
| 35g | 1-(4-(4-(morpholinopropyl)-1H-1,2,3-triazol-1-yl)phenyl)-3-o-tolylurea |
| 35f | 1-(4-(4-(morpholinoethyl)-1H-1,2,3-triazol-1-yl)phenyl)-3-o-tolylurea |
| 35e | 1-(4-(4-(morpholinomethyl)-1H-1,2,3-triazol-1-yl)phenyl)-3-o-tolylurea |
| 35h | 4-(4-(3-(4-methylpiperazin-1-yl)propyl)-1H-1,2,3-triazol-1-yl)benzenamine |
| 8d | 1-(4-(4-hexyl-1H-1,2,3-triazol-1-yl)phenyl)-3-(2-(trifluoromethyl)phenyl)urea) |
| 42b | 1-(4-(4-(3-fluoropropyl)-1H-1,2,3-triazol-1-yl)phenyl)-3-(2 |
| | (trifluoromethyl) phenyl) urea |
| 21b | 1-(3-chloro-2-methylphenyl)-3-(4-(4-isopentyl-1H-1,2,3-triazol-1-yl)phenyl)urea |
| 20d | 3-(1-(4-(3-(2-(trifluoromethyl)phenyl)ureido)phenyl)-1H-1,2,3-triazol-4- |
| | yl)propanoic acid |
| 55c | 1-(4-fluorophenyl)-3-(4-(4-isopentyl-1H-1,2,3-triazol-1-yl)phenyl)urea |
| 35e | 1-(4-(4-(morpholinomethyl)-1H-1,2,3-triazol-1-yl)phenyl)-3-o-tolylurea |
| 42c | 1-(4-(4-(1-fluoro-2-methylpentyl)-1H-1,2,3-triazol-1-yl)phenyl)-3-(2- |
| | (trifluoromethyl) phenyl) urea |
| 78 | 4-(4-isopentyl-1H-1,2,3-triazol-1-yl)-3-methoxyaniline |
| 22g | 1-(4-(4-(((3,4-dihydroxy-5-(hydroxymethyl)tetrahydrofuran-2- |
| | yl)oxy)methyl)-1H-1,2,3-triazol-1-yl)phenyl)-3-(2-(trifluoromethyl)phenyl)urea |
| 35d | 1-(4-(4-(3-hydroxyhexan-2-yl)-1H-1,2,3-triazol-1-yl)phenyl)-3-(2- |
| | (trifluoromethyl)phenyl) urea |
| 55l | 1-(4-(4-butyl-1H-1,2,3-triazol-1-yl)phenyl)-3-(3-fluorophenyl)urea |
| 55i | 1-(4-(4-butyl-1H-1,2,3-triazol-1-yl)phenyl)-3-(5-(methoxymethoxy)-2-methylphenyl)urea |
| 55h | 1-(4-(4-butyl-1H-1,2,3-triazol-1-yl)phenyl)-3-(5-(cyclopentyloxy)-2-methylphenyl)urea |
| 55o | 1-(4-(4-(ethoxymethyl)-1H-1,2,3-triazol-1-yl)phenyl)-3-(3-fluorophenyl)urea |
| 55n | 1-(3-fluorophenyl)-3-(4-(4-(3-oxobutyl)-1H-1,2,3-triazol-1-yl)phenyl)urea |
| 102 | 1-(4-(5-butylisoxazol-3-yl)phenyl)-3-(2-(trifluoromethyl)phenyl)urea |
| 42a | 1-(4-(4-(fluoromethyl)-1H-1,2,3-triazol-1-yl)phenyl)-3-(2-(trifluoromethyl)phenyl)urea |
| 55p | 1-(4-(4-butyl-1H-1,2,3-triazol-1-yl)phenyl)-3-(2-methyl-5-(3-oxobutyl)phenyl)urea |
| 55q | 1-(4-(4-butyl-1H-1,2,3-triazol-1-yl)phenyl)-3-(5-fluoropyridin-3-yl)urea |
| 64c | 3-(4-butyl-1H-1,2,3-triazol-1-yl)-N-(o-tolyl)benzenesulfonamide |
| 130 | 2-butyl-5-(4-nitrophenyl)-1,3,4-oxadiazole |
| 65c | 4-(4-butyl-1H-1,2,3-triazol-1-yl)-N-(2- |
| | (trifluoromethyl)phenyl)benzenesulfonamide |
| 67d | 3-(4-isopentyl-1H-1,2,3-triazol-1-yl)-N-(2 (trifluoromethyl)phenyl) benzene sulfonamide |
| 58d | N-(2-methyl)-3-nitro-phenylenzenesulfonamide |
| 66e | 3-(4-(2-methoxyethyl)-1H-1,2,3-triazol-1-yl)-N-(2- |
| | (trifluoromethyl)phenyl)benzenesulfonamide |
| 58c | N-(2-trifluoromethyl)-3-nitro-phenylbenzenesulfonamide |
| 59a | N-(2-hydroxy)-4-nitro-phenylbenzenesulfonamide |
| 59d | N-(2-methyl)-4-nitro-phenylenzenesulfonamide |
| 71 | 3-(2H-tetrazol-5-yl)-N-(2-(trifluoromethyl)phenyl)benzenesulfonamide |
| | benzenesulfonamide potassium salt |

TABLE 13
DDX3 inhibitors from Samal et al., 2015

| Commercial | |
|---|---|
| Name | Compound name |
| Toradol | Ketorolac tromethamine OR |
| | (+/−)-5-Benzoyl-2,3-dihydro-1H-pyrrolizine-1-carboxylic |
| | acid tris (hydroxymethyl) amino methane salt |

TABLE 14
DDX3 inhibitors from WO2015200779 and WO 2010/039187

| | |
|---|---|
| 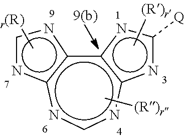 | Formula (I) |

(I) or pharmaceutically acceptable salts and prodrugs thereof, wherein:
R, R′, and R″ are each independently a hydrogen, hydroxyl; substituted or unsubstituted: cyclic and acyclic alkyl group, cyclic and acyclic alkenyl group, cyclic and acyclic alkynyl group, aryl group, alkylaryl group, arylalkyl group, benzyl group, cyclic and acyclic heteroalkyl group, heteroaryl group; —C(O)R³; —C(S)R³; ¹S(O)R³; —S(O)₂R³; —C(O)NR⁴R⁵; —C(S)NR³R⁴; —C(X)YR⁵R⁶; -β-D-ribosyl; -α-D-ribosyl; -β-L-ribosyl; -α-L-ribosyl; T-deoxy-β-D-ribosyl; 2-deoxy-β-L-ribosyl; 2′-deoxy-α-D-ribosyl; 2′-deoxy-α-L-ribosyl; and ribose or deoxyribose sugars substituted with one or more halogens;
R, R′, and R″ can also form a ring with one or more C, S, O, N atoms such that, for example,
R and R′ together include:
R⁷

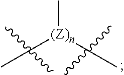

R⁷ is a hydrogen; hydroxyl; substituted and unsubstituted: cyclic and acyclic alkyl group, group, alkenyl group, alkynyl group, aryl group, aryloxy group, alkylary group, aryalkyl group, heteroaryl group, heterocycloalkyl group; —C(O)alkyl; —C(O)alkenyl; —C(O)alkynyl; —C(O)aryl; —C(O)benzyl; —C(O)NR³R⁴; —C(S)alkyl; —C(S)alkenyl; —C(S)alkynyl; —C(S)aryl; —C(S)benzyl; —C(S)NR³R⁴; —C(X)YR¹R²; wherein
Q is O, NH, or S;
X is O, N, or S;
Y is O, CH₂, NH, or S; Z is CH, N, P, or C; is a single bond or double bond; wherein if is a double bond, R² or R⁷ is independently O, S, or NH; n is 1, 2, 3, or 4; and r, r′, and r″ are each independently an integer from 1 to about 3.

In certain illustrative embodiments, R, R′, and R″ are each independently a substituted benzyl, alkyl, aryl, cycloalkyl, heteroaryl, or heterocycloalkyl with one or more substituents, such as, but not limited to, —H, —F, —Cl, —Br, —I, —OH, azido, —SH, alkyl, aryl, heteroalkyl, alkyoxyl, alkylthiol, amino, hydroxylamino, N-alkylamino, —N,N-dialkylamino, —N,N-dimethylamino, acyl, alkyloxycarbonyl, sulfonyl, urea, —NO₂, triazolyl.

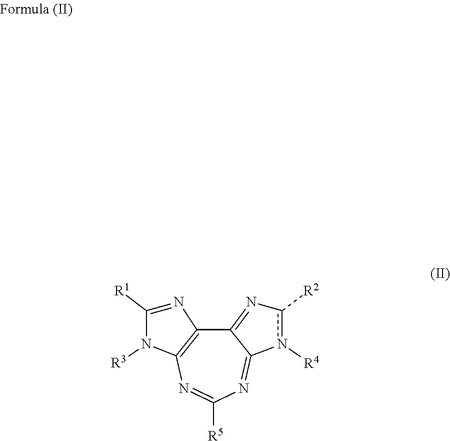

or pharmaceutically acceptable salts and prodrugs thereof, wherein
R¹, R², R³, R⁴, and R⁵ are each independently a hydrogen; hydroxyl; substituted or unsubstituted: cyclic and acyclic alkyl group, cyclic and acyclic alkenyl group, cyclic and acyclic alkynyl group, aryl group, alkylaryl group, arylalkyl group, benzyl group, cyclic and acyclic heteroalkyl group, heteroaryl group; —C(O)R³; —C(S)R³; —S(O)R³; —S(O)₂R³;
—C(O)NR⁴R⁵; —C(S)NR³R⁴; —C(X)YR⁵R⁶; -β-D-ribosyl; -α-D-ribosyl; —O-L-ribosyl; -α-L-ribosyl; 2′-deoxy-β-D-ribosyl; 2-deoxy-β-L-ribosyl; 2-deoxy-α-D-ribosyl; 2′-deoxy-α-L-ribosyl; and ribose or deoxyribose sugars substituted with one or more halogens; R¹ and R³ or R² and R⁴ can also form a ring with one or more C, S, O, N atoms such that R¹ and R³ or R² and R⁴ together include

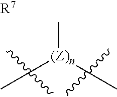

R⁷ is a hydrogen; hydroxyl; substituted and unsubstituted: cyclic and acyclic alkyl group, group, alkenyl group, alkynyl group, aryl group, aryloxy group, alkylary group, aryalkyl group, heteroaryl group, heterocycloalkyl group; —C(O)alkyl; —C(O)alkenyl; —C(O)alkynyl; —C(O)aryl; —C(O)benzyl; —C(O)NR³R⁴; —C(S)alkyl; —C(S)alkenyl; —C(S)alkynyl; —C(S)aryl; —C(S)benzyl; —C(S)NR³R⁴; —C(X)YR¹R²; wherein X is O, N, or S; Y is O, CH₂, NH, or S; Z is CH, N, P, or C, is a single bond or double bond; wherein if is a double bond, R² or R⁷ is independently O, S, or NH; and n is 1, 2, 3, or 4.

In certain illustrative embodiments, R¹, R², R³, R⁴, and R⁵ are each independently are a substituted benzyl, alkyl, aryl, cycloalkyl, heteroaryl, or heterocycloalkyl with one or more substituents, such as, but not limited to, —H, —F, —Cl, —Br, —I, —OH, azido, —SH, alkyl, aryl, heteroalkyl, alkyoxyl, alkylthiol, amino, hydroxylamino, N-alkylamino, —N,N-dialkylamino, —N,N-dimethylamino, acyl, alkyloxycarbonyl, sulfonyl, urea, —NO₂, triazolyl.

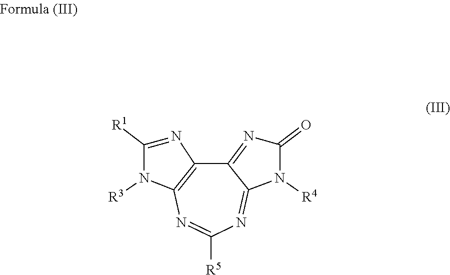

or pharmaceutically acceptable salts and prodrugs thereof, wherein
R¹, R³, R⁴, and R⁵ are each independently a hydrogen; hydroxyl; substituted or unsubstituted: cyclic and acyclic alkyl group, cyclic and acyclic alkenyl group, cyclic and acyclic alkynyl group, aryl group, alkylaryl group, arylalkyl group, benzyl group, cyclic and acyclic heteroalkyl group, heteroaryl group; —C(O)R³; —C(S)R³; —S(O)R³; —S(O)₂R³; —C(O)NR⁴R⁵; —C(S)NR³R⁴; —C(X)YR⁵R⁶; -β-D-ribosyl; -α-D-ribosyl; -β-L-ribosyl; -α-L-ribosyl; 2′-deoxy β-D-ribosyl; 2′-deoxy-β-L-ribosyl; 2′-deoxy-α-D-ribosyl; 2-deoxy-α-L-ribosyl; and ribose or deoxyribose sugars substituted with one or more halogens;
R¹ and R³ can also form a ring with one or more C, S, O, N atoms such that R¹ and R³ together include

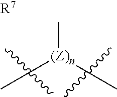

R⁷ is a hydrogen; hydroxyl; substituted and unsubstituted: cyclic and acyclic alkyl group, group, alkenyl group, alkynyl group, aryl group, aryloxy group, alkylary group, aryalkyl group, heteroaryl group, hetercyeloalkyl group; —C(O)alkyl; —C(O)alkenyl; —C(O)alkynyl; —C(O)aryl; —C(O)benzyl; —C(O)NR³R⁴; —C(S)alkyl; —C(S)alkenyl; —C(S)alkynyl; —C(S)aryl; —C(S)benzyl; —C(S)NR³R⁴; —C(X)YR¹R²; wherein
X is O, N, or S;
Y is O, CH₂, NH, or S;
Z is CH, N, P, or C; is a single bond or double bond; wherein if is a double bond, R² or R⁷ is independently O, S, or NH; and n is 1, 2, 3, or 4.

In certain illustrative embodiments, R¹, R³, R⁴, and R⁵ are not all hydrogen. In certain illustrative embodiments, R¹, R³, R⁴, and R⁵ are each independently are a substituted benzyl, alkyl, aryl, cycloalkyl, heteroaryl, or heterocycloalkyl with one or more substituents, such as, but not limited to, —H, —F, —Cl, —Br, —I, —OH, azido, —SH, alkyl, aryl, heteroalkyl, alkyoxyl, alkylthiol, amino, hydroxylamino, N-alkylamino, —N,N-dialkylamino, —N,N-dimethylamino, acyl, alkyloxycarbonyl, sulfonyl, urea, —NO₂, triazolyl.

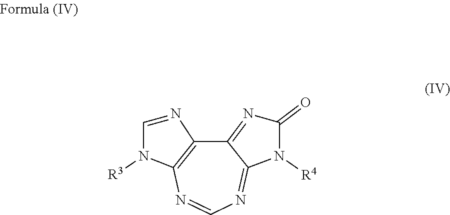

or pharmaceutically acceptable salts and prodrugs thereof, wherein
R³ and R⁴ are each independently a hydrogen; hydroxyl; substituted or unsubstituted: cyclic and acyclic alkyl group, cyclic and acyclic alkenyl group, cyclic and acyclic alkynyl group, aryl group, alkylaryl group, arylalkyl group, benzyl group, cyclic and acyclic heteroalkyl group, heteroaryl group; —C(O)R³; —C(S)R³; —S(O)R³; —S(O)₂R; —C(O)NR⁴R⁵; —C(S)NR³R⁴; —C(X)YR⁵R⁶; -β-D-ribosyl; -α-D-ribosyl; -β-L-ribosyl; -α-L-ribosyl; T-deoxy-β-D-ribosyl; 2-deoxy-β-L-ribosyl; 2′-deoxy-α-D-ribosyl; 2′-deoxy-α-L-ribosyl; and ribose or deoxyribose sugars substituted with one or more halogens.

In certain illustrative embodiments, R³ and R⁴ are both not hydrogen.

In certain illustrative embodiments, R and R⁴ are each independently are a substituted benzyl, alkyl, aryl, cycloalkyl, heteroaryl, or heterocycloalkyl with one or more substituents, such as, but not limited to, —H, —F, —Cl, —Br, —I, —OH, azido, —SH, alkyl, aryl, heteroalkyl, alkyoxyl, alkylthiol, amino, hydroxylamino, N-alkylamino, —N,N-dialkylamino, —N,N-dimethylamino, acyl, alkyloxycarbonyl, sulfonyl, urea, —NO₂, triazolyl.

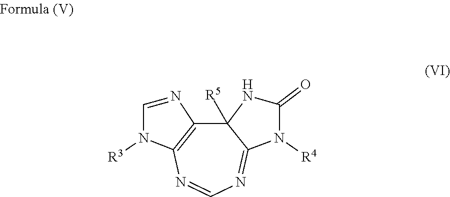

or pharmaceutically acceptable salts and prodrugs thereof, wherein
R³, R⁴, and R⁵ are each independently a hydrogen; hydroxyl; substituted or unsubstituted: cyclic and acyclic alkyl group, cyclic and acyclic alkenyl group, cyclic and acyclic alkynyl group, aryl group, alkylaryl group, arylalkyl group, benzyl group, cyclic and acyclic heteroalkyl group, heteroaryl group; —C(O)R³; —C(S)R³; —S(O)R³; —S(O)₂R³; —C(O) NR⁴R⁵; —C(S)NR³R⁴; —C(X)YR⁵R⁶; -β-D-ribosyl; -α-D-ribosyl; -β-L-ribosyl; -α-L-ribosyl; T-deoxy-β-D-ribosyl; 2′-deoxy-β-L-ribosyl; 2′-deoxy-α-D-ribosyl; 2′-deoxy-α-L,-ribosyl; and ribose or deoxyribose sugars substituted with one or more halogens. In certain illustrative embodiments, R³, R⁴, and R⁵ are not each hydrogen. In certain illustrative embodiments, R³, R⁴, and R⁵ are each independently are a substituted benzyl, alkyl, aryl, cycloalkyl, heteroaryl, or heterocycloalkyl with one or more substituents, such as, but not limited to, —H, —F, —Cl, —Br, —I, —OH, azido, —SH, alkyl, aryl, heteroalkyl, alkyoxyl, alkylthiol, amino, hydroxylamino, N-alkylamino, —N,N-dialkylamino, —N,N-dimethylamino, acyl, alkyloxycarbonyl, sulfonyl, urea, —NO₂, triazolyl.

TABLE 15
DDX3 inhibitors from Yedavalli et al., 2008

| Reference | Commercial | |
|---|---|---|
| Name | Name | Compound name |
| Compound 1 | | 4,5-Dihydro-8H-6-(N-octadecyl)amino- |
| | | 1-(β-D-ribofuranosyl)imidazo[4,5- |
| | | e]diazepine-4,8-dione |
| Compound 2 | NZ51 or | 4,5-Dihydro-8H-6-(N- |
| | NZ-51 | hexadecyl)amino-1-(2′-deoxy-α-D- |
| | | erythropentofuranosyl)imidazo[4,5- |
| | | e]diazepine-4,8-dione |

TABLE 16
DDX3 inhibitors from Maga et al., 2008

| | | | 1-14 |
|---|---|---|---|
| 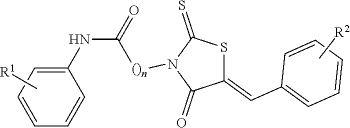 | | | |
| | | | 15-21 |
| 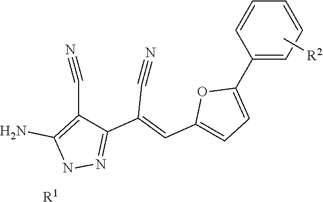 | | | |
| Compound | R¹ | R² | n |
| 1 | 3-OH | 2-OCH₃ | 2 |
| 2 | 3-OH | 3-Br | 2 |
| 3 | 4-OH | 3-Br | 2 |
| 4 | 2-OH | 3-Br | 2 |
| 5 | 3-OH | 3-Br | 3 |
| 8 | 2-OH | 3-Br | 3 |
| 13 | 2-COOH | 3-Br | 2 |
| 15 | Ph | 2-NO₂ | — |
| 17 | H | 3-NO₂ | — |
| 18 | Ph | 3-Cl | — |

TABLE 17a
DDX3 inhibitors from Maga et al., 2011

| | | | 4 |
|---|---|---|---|
| 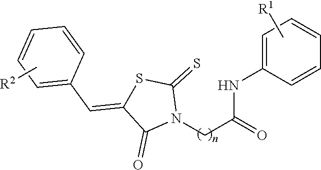 | | | |
| | | | 7 |
| 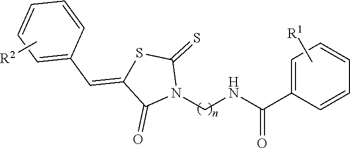 | | | |
| | | | 11 |
| 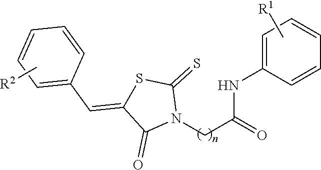 | | | |
| | | | 31 |
| 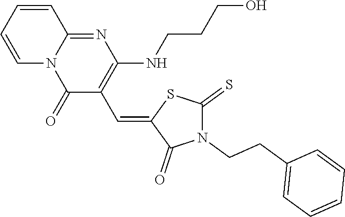 [SMILES: O=C1/C(=C/c2c(NCCCO)nc3ccccn3c2=O)SC(=S)N1CCc1ccccc1] | | | |
| Compound | R¹ | R² | n |
| 4a | 2-OH | 3-Br | 2 |
| 4b | 2-OH | 3-F | 2 |
| 4c | 2-OH | 3,5-diF | 2 |
| 4d | 2-OH | 3-Me | 2 |
| 4e | 2-OH | 3-OMe | 2 |
| 4f | 2-OH | 3-(O—CH₂—O)-4 | 2 |
| 4g | 2-NO₂ | 3-Br | 2 |
| 4h | 2-NO₂ | 3-F | 2 |
| 4l | 2-OH | 3,4,5-triOMe | 2 |
| 4m | 2-Cl | 3,4,5-triOMe | 2 |
| 4p | 2-OH, 5-Cl | 4-OMe | 2 |
| 4r | 2-COOH | 3-Br | 2 |
| 4s | 2-COOH | 3-(O—CH₂—O)-4 | 2 |
| 4t | 2-COOH | 4-OMe | 2 |
| 4u | 3-OH | 3-Br | 2 |
| 4v | 3-OH | 2-OMe | 2 |
| 4w | 4-OH | 3-Br | 2 |
| 4x | 2-OH | 3-Br | 3 |
| 4z | 3-OH | 3-Br | 3 |
| 7a | 2-OH | 3-Br | 2 |
| 7b | 2-OH | 3-F | 2 |
| 11 | 2-OH | 3-br | 2 |
| 31 | — | — | — |

TABLE 17b
DDX3 inhibitors from Maga et al., 2011

| | | | | 15a-g |
|---|---|---|---|---|
| 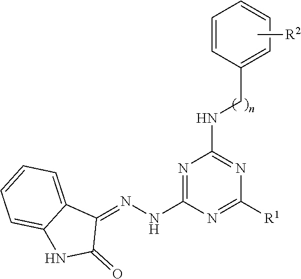 | | | | |
| | | | | 16a-p |
| 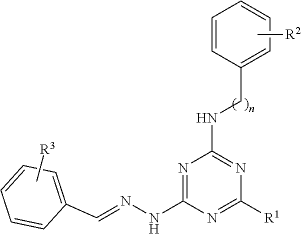 | | | | |
| | | | | 20a-c |
| 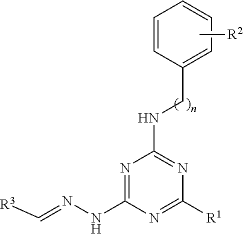 | | | | |
| | | | | 21a-d |
| 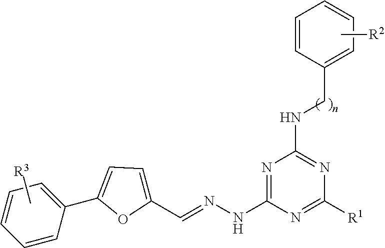 | | | | |
| | | | | 30 |
| 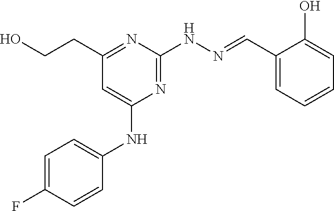 [SMILES: OCCc1cc(Nc2ccc(F)cc2)nc(N/N=C/c2ccccc2O)n1] | | | | |
| Compound | R¹ | R² | R³ | n |
| 15a | NH—Ph | H | — | 0 |
| 15c | morpholinyl | 3-Cl | — | 0 |
| 15d | morpholinyl | 3-Cl, 4-Me | — | 0 |
| 15f | morpholinyl | 3-Cl | — | 1 |
| 15g | NEt₂ | H | — | 1 |
| 16a | NH—Ph | H | 2,4-OH | 0 |
| 16b | NH—Ph | H | 3,4-diCl | 0 |
| 16c | NH—Ph | H | 3-NO₂, 4-OH | 0 |
| 16e | NH—Ph | H | 4-NHCOCH₃ | 0 |
| 16f | NH—Ph | H | 4-NHCOCH₃ | 1 |
| 16g | morpholinyl | H | 4-NHCOCH₃ | 0 |
| 16h | morpholinyl | 3-Cl, 4-Me | 2-OH | 0 |
| 16i | morpholinyl | 4-F | 2-OH | 1 |
| 16j | morpholinyl | H | 2-OH | 1 |
| 16k | morpholinyl | H | 2-OH, 5-Cl | 1 |
| 16l | morpholinyl | H | 2-OH, 3-NO₂ | 1 |
| 16m | morpholinyl | 4-F | 2-OH | 0 |
| 16n | NH—Ph(4-F) | 4-F | 2-OH | 0 |
| 16o | morpholinyl | 3-Cl | 2-OH | 1 |
| 16p | NEt₂ | H | 2-OH | 1 |
| 20a | morpholinyl | 4-F | 2-methyl-indol- | 0 |
| | | | 3-yl | |
| 20b | morpholinyl | 4-F | 1-naphthyl | 0 |
| 20c | morpholinyl | 4-F | 2-naphthyl | 0 |
| 21a | morpholinyl | 4-F | 2-Cl, 4-NO₂ | 0 |
| 21b | morpholinyl | 4-F | 2-Cl, 5-NO₂ | 0 |
| 21c | morpholinyl | H | 2-Cl | 0 |
| 21d | piperidinyl | H | 4-Cl | 0 |
| 30 | — | — | — | — |

The following examples illustrate aspects of the invention; no example is intended to encompass the invention as a whole. Furthermore, although some examples may present discrete embodiments of the invention, aspects of such examples may be combined with other variations of the invention as described above or in different examples unless such combinations would be clearly inoperable to one of skill in the art.

### Example 1: DDX3 Inhibitors Block the NEU3 Up-Regulation Induced by TGF-β1 or IL-6 in Human Small Airway Epithelial Cells (HSAEpCs)

High levels of the released active form of TGF-β1 are often observed in specific tissues in diseases such as cancer, fibrosis and inflammation. IL-6 is elevated in the serum, BAL fluid, and lung tissue in patients with, and animal models of, pulmonary fibrosis. High levels of NEU3 are observed in human and mouse pulmonary fibrosis. Mice lacking NEU3 show attenuated pulmonary fibrosis, and aspiration of NEU3 induces pulmonary fibrosis. Both TGF-β1 and IL-6 increase NEU3 expression in human PBMCs. Attenuated pulmonary fibrosis in sialidase-3 knockout (Neu3−/−) mice, American Journal of Physiology-Lung Cellular and Molecular Physiology, 2020, 318(1): L165-L179. Conversely, NEU3 upregulates TGF-β1. Together, these observations suggest that inhibiting NEU3 up-regulation induced by TGF-β1 or IL-6 could attenuate fibrosis.

To determine if DDX3 mediates the regulation effect of TGF-β1 or IL-6 on NEU3, HSAEpCs were treated with or without 1 or 10 μM RK33 (Selleckchem, Radnor, PA) from a 10 mM stock in dimethylsulfoxide (DMSO) (VWR, Radnor, PA), and 0.1, 1 or 10 μM IN-1 (AdooQ, Irvine, CA) from a 10 mM stock in DMSO in the presence or absence of 10 ng/ml TGF-β1 (PeproTech, Rocky Hill, NJ) from a 50 g/ml stock in PBS or 1 ng/ml IL-6 (BioLegend, San Diego, CA) from a 200 g/ml stock in 10 mM NaH2PO4, 150 mM NaCl, pH 7.2. After 48 hours, the culture medium was discarded and cells were rinsed with PBS three times at room temperature. Cells were detached with 0.5 ml Accutase (Innovative Cell Technologies, San Diego, CA) for 5 minutes at 37° C., then cells were collected by centrifugation at 300×g for 5 minutes and resuspended in 1 ml PBS. Cells were counted and 1×10⁶ cells were re-collected by centrifugation. The cell pellet was lysed in 200 μl RIPA buffer (Thermo Scientific, Rockford, IL) containing protease/phosphatase inhibitor (Cell Signaling Technology, Danvers, MA) on ice for 30 minutes. The mixture was clarified by centrifugation at 12,000×g for 15 minutes at 4° C. 80 μl of supernatant was mixed with 20 μl 5×SDS sample buffer and heated at 95° C. for 5 minutes. The mixture was used for Coomassie blue staining and western blotting. Western blots were performed as described in Experimental lung research, 2020, 46(3-4): 75-80 using rabbit anti-NEU3 (NBP3-04868, NOVUS Biologicals, Centennial, CO).

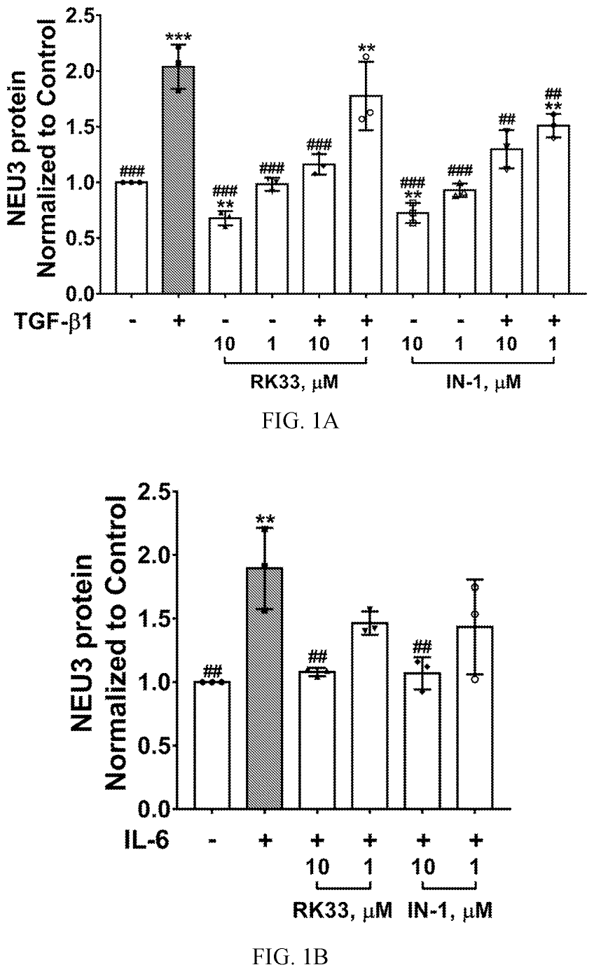

Results of NEU3 expression in HSAEpCs are presented in FIGS. 1A-1B. Compared to control, TGF-β1 and IL-6 increased NEU3 significantly. 10 μM DDX3 inhibitors blocked the up-regulation of NEU3 induced by TGF-β1 and IL-6.

### Example 2: DDX3 siRNA Blocks the NEU3 Up-Regulation Induced by TGF-β1 in Human Lung Fibroblasts (HLFs)

To determine if DDX3 mediates the regulation effect of TGF-β1 on NEU3, HLFs were transfected with DDX3 siRNA (#4392420, Thermo Scientific, Rockford, IL) or negative control siRNA (#4390843, Thermo Scientific), following Optimization of transfection conditions and analysis of siRNA potency using real-time PCR, Therapeutic Oligonucleotides, 2011, 199-213. 24 hours later, HLFs were treated with or without 10 ng/ml TGF-β1. After 48 hours, the culture medium was discarded and cells were lysed and collected as described in example 1. Western blots were performed as described in example 1 with rabbit anti-DDX3 (NBP1-85291, NOVUS) and rabbit anti-NEU3 (NBP3-04868, NOVUS).

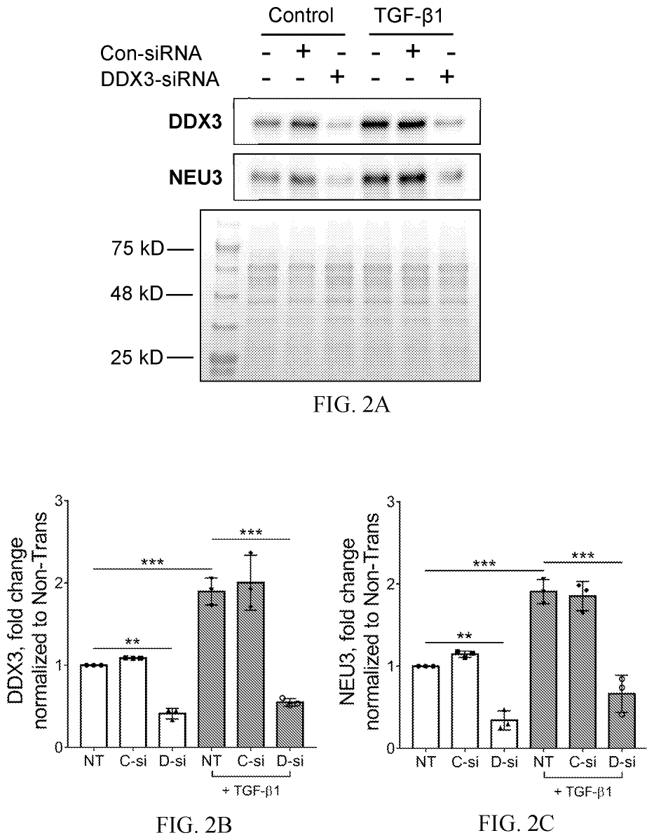

Results of DDX3 and NEU3 expression in transfected or non-transfected HLFs are presented in FIGS. 2A-2C. Compared to non-transfected cells, DDX3 siRNA decreased DDX3 and NEU3 while TGF-β1 increased DDX3 and NEU3 levels. DDX3 siRNA also blocked the TGF-β1 induced DDX3 and NEU3 upregulation. Negative control siRNA did not decrease DDX3 or NEU3. These data suggest that DDX3 mediates the regulation effect of TGF-β1 on NEU3.

### Example 3: DDX3 Inhibitor Increases Survival Rates and Alleviates Fibrosis of Bleomycin Treated Mice

To determine if the DDX3 inhibitor affects fibrotic symptoms in mice, 7 to 8 week-old C57BL/6 mice (Jackson, Bar Harbor, ME) were treated with an oropharyngeal aspiration of 50 μl of 0.9% saline, or 5 U/kg bleomycin (Calbiochem, EMD Millipore, Billerica, MA) in 50 μl of 0.9% saline, as described in Oropharyngeal aspiration of a silica suspension produces a superior model of silicosis in the mouse when compared to intratracheal instillation, Experimental Lung Research, 2006, 32:181-199. The successful aspiration of bleomycin into the lungs was confirmed by listening for a crackling noise heard after the aspiration. 100 μl DMSO or 20 mg/kg RK33 in 100 μl DMSO was given to mice by intraperitoneal (IP) injection at day 10, 12, 14, 16, 18 and 20 after bleomycin treatment. Mice were weighed daily, and euthanized using CO2 following NIH guidelines at day 21 after bleomycin aspiration. The experiment was performed in accordance with the recommendations in the Guide for the Care and Use of Laboratory Animals of the National Institutes of Health. The Texas A&M University Animal Use and Care Committee approved the protocol.

After euthanasia, the lungs were perfused three times with 300 microliters PBS, pH 7.4, to collect bronchoalveolar lavage fluid (BAL) as described in Oropharyngeal aspiration of a silica suspension produces a superior model of silicosis in the mouse when compared to intratracheal instillation, Experimental Lung Research, 2006, 32:181-199. The BALs were clarified by centrifugation at 500×g for 10 minutes, and the supernatants were transferred to Eppendorf tubes. The cells collected from BAL were counted, and BAL cytospins were prepared as described in Fibroblasts secrete Slit2 to inhibit fibrocyte differentiation and fibrosis, Proc Natl Acad Sci USA, 2014, 111: 18291-18296. BAL cytospins were fixed and treated with Wright-Giemsa stain (#08711, Polysciences, Inc., Warrington, PA) following Variation of differential cell counts of bronchoalveolar lavage fluid, Archives of pathology & laboratory medicine, 1986, 110(4): 341-343.

After collection of BAL fluid, mouse lungs were inflated with prewarmed Optimal Cutting Temperature (OCT) embedding medium (VWR, Radnor, PA) and then embedded in OCT, frozen on dry ice, and then stored at −80° C. 8-micron cryosections of mouse lungs were mounted on Superfrost Plus® microscope slides (VWR). The sections on the slides were allowed to air-dry for 24-48 hours. Total collagen on the mouse lung sections were stained with picrosirius red as described in Reduction of bleomycin-induced pulmonary fibrosis by serum amyloid P, J Immunol, 2007, 179: 4035-4044. Hydroxyproline in the mouse lung tissue was measured with a hydroxyproline assay as described in Attenuated pulmonary fibrosis in sialidase-3 knockout (Neu3−/−) mice, Am J Physiol Lung Cell Mol Physiol, 2020, 318(1): L165-L179.

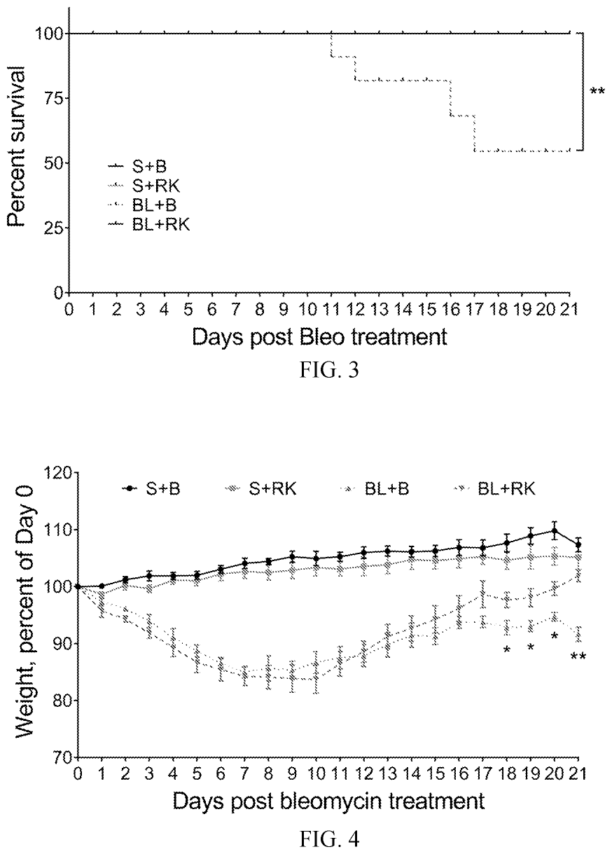

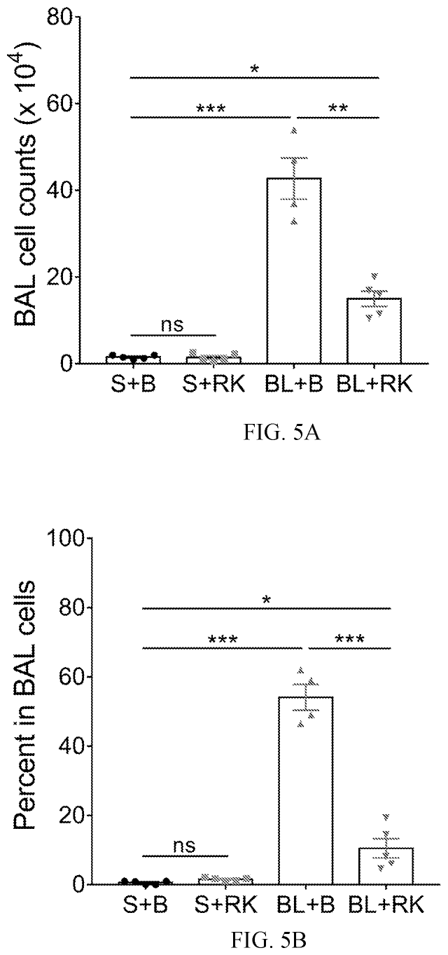

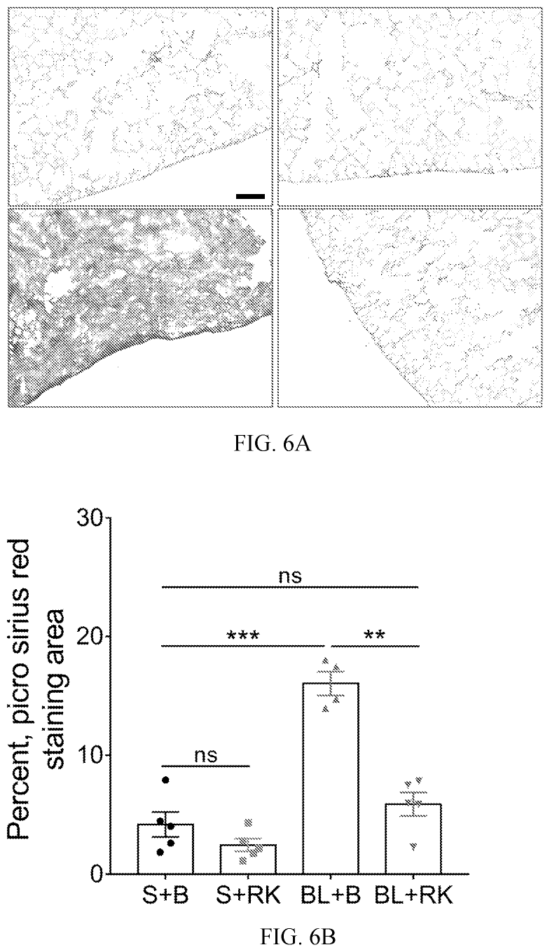

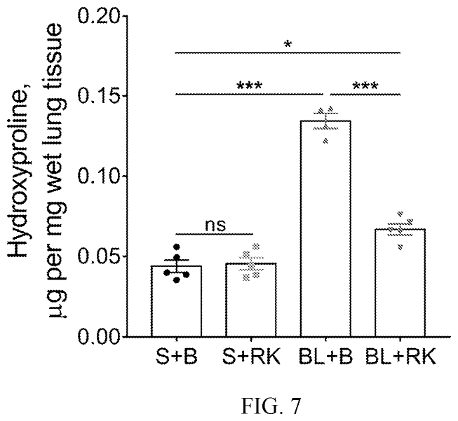

As shown in FIG. 3 and FIG. 4, RK33 dramatically increased survival rate and improved the body weight recovery of the bleomycin treated mice. The effect of RK33 on accumulation of immune cells in BAL is presented in FIGS. 5A-5B. Bleomycin increased the total cell number and percentage of lymphocytes in BAL, and RK33 significantly blocked the bleomycin effect. As shown in FIGS. 6A-6B, total collagen was increased in the lungs of bleomycin+buffer treated mice, and RK33 reduced the abnormally high levels of collagen in bleomycin treated mouse lungs. There was no significant difference between the lung of bleomycin+RK33 treated mouse and the control mouse. As shown in FIG. 7, total collagen was increased in the lungs of bleomycin+buffer treated mice, and RK33 reduced the abnormally high levels of collagen in bleomycin treated mouse lungs. Together, these results indicate that DDX3 inhibitors can attenuate fibrosis.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

1-32. (canceled)

33. A method of suppressing fibrosis in a mammal having a fibrosing disease and/or fibrotic condition, comprising administering one or more compounds that inhibits the activity of ATPase/RNA helicase X-linked DEAD-box polypeptide 3 (DDX3), or a pharmaceutical composition comprising said one or more compounds, to the mammal.

34. The method of claim 33, wherein said one or more compound, or a pharmaceutical composition comprising said one or more compound, is administered in an amount sufficient to suppress fibrosis in the mammal.

35. The method of claim 33, wherein said one or more compound or said pharmaceutical composition comprises a compound of Tables 1-16, 17A, or 17B.

36. The method of claim 35, wherein said compound is diimidazo[4,5-d:4′,5′-f]-[1,3]diazepine (RK-33).

37. The method of claim 33, said method further comprising administering to said mammal serum amyloid P (SAP) and/or fragments thereof, IL-12, Laminin-1, IgG aggregates, cross-linked IgG, cofactors of any of the above, any combination or a pharmaceutical composition comprising serum amyloid P (SAP) and/or fragments thereof, IL-12, Laminin-1, IgG aggregates, cross-linked IgG, cofactors of any of the above, or any combination thereof.

38. The method of claim 37, said fragments of SAP being selected from the group consisting of KERVGEYSLYIGRHKVTSKVIEKFP (SEQ ID NO:1), ILSAYQGTPLPA (SEQ ID NO:2), IRGYVIIKPLV (SEQ ID NO:3), and combinations thereof.

39. The method of claim 33, wherein the mammal has a fibrosing disease and/or fibrotic condition selected from keloid scarring, rheumatoid arthritis, lupus, nephrogenic fibrosing dermopathy, fibrotic lesions such as those formed after Schistosoma japonicum infection, autoimmune diseases, Lyme disease, stromal remodeling in pancreatitis and stromal fibrosis, chronic obstructive pulmonary disease, pulmonary fibrosis, uterine fibroids, ovarian fibrosis, other fibrocystic formations, corneal fibrosis or other eye fibrosis, such as that resulting from corneal refraction surgery, fibrosis resulting from congestive heart failure and other post-ischemic conditions, abdominal adhesions, wide angle glaucoma trabeculotomy, and any combinations thereof.

40. The method of claim 33, said fibrosing disease and/or fibrotic condition:
a) being fibrosis of the liver that results from alcohol, drug, and/or chemically induced cirrhosis, ischemia-reperfusion injury after hepatic transplant, necrotizing hepatitis, hepatitis B, hepatitis C, primary biliary cirrhosis, and primary sclerosing cholangitis;
b) being fibrosis arising in the kidney that results from: proliferative and sclerosing glomerulonephritis, nephrogenic fibrosing dermopathy, diabetic nephropathy, renal tubulointerstitial fibrosis, and focal segmental glomerulosclerosis;
c) being fibrosis that is associated with the lungs arising from: pulmonary interstitial fibrosis, sarcoidosis, pulmonary fibrosis, idiopathic pulmonary fibrosis, asthma, chronic obstructive pulmonary disease, diffuse alveolar damage disease, pulmonary hypertension, neonatal bronchopulmonary dysplasia, chronic asthma, and emphysema;
d) being fibrosis of the heart and/or pericardium that is selected from myocardial fibrosis, atherosclerosis, coronary artery restenosis, congestive cardiomyopathy, heart failure, and other post-ischemic conditions;
e) being a fibrosing disease of the eye, selected from exophthalmos of Grave's disease, proliferative vitreoretinopathy, anterior capsule cataract, corneal fibrosis, corneal scarring due to surgery, trabeculectomy-induced fibrosis, progressive subretinal fibrosis, multifocal granulomatous chorioretinitis, and other eye fibrosis;
f) being a fibrosing disease of the skin selected from: Dupuytren's contracture, scleroderma, keloid scarring, psoriasis, hypertrophic scarring due to burns, atherosclerosis, restenosis, and psuedoscleroderma caused by spinal cord injury;
g) being fibrotic diseases of the mouth and/or esophagus selected from periodontal disease scarring, gingival hypertrophy secondary to drugs, and congenital esophageal stenosis;
h) being fibrotic diseases affecting the pancreas selected from pancreatic fibrosis, stromal remodeling pancreatitis, and stromal fibrosis;
i) being fibrotic diseases affecting the gastrointestinal tract selected from collagenous colitis, villous atrophy, crypt hyperplasia, polyp formation, fibrosis of Crohn's disease, and healing gastric ulcer;
j) being fibrotic conditions affecting the brain selected from glial scar tissue;
k) being a fibrotic condition of the breast selected from fibrocystic disease of the breast and desmoplastic reaction to breast cancer;
l) being fibrosis arising in the bone marrow selected from fibrosis in myelodysplasia and neoplastic diseases and rheumatoid pannus formation;
m) being fibrotic diseases/conditions of the genitourinary system selected from endometriosis, uterine fibroids, ovarian fibroids, and Peyronie's disease; or
n) being fibrosis caused by radiation induced damage used to treat various forms of cancer selected from head and neck cancer, ovarian cancer, prostate cancer, lung cancer, gastrointestinal cancer, colon cancer, and breast cancer.

41. The method of claim 37, wherein the mammal has a fibrosing disease and/or fibrotic condition selected from keloid scarring, rheumatoid arthritis, lupus, nephrogenic fibrosing dermopathy, fibrotic lesions such as those formed after Schistosoma japonicum infection, autoimmune diseases, Lyme disease, stromal remodeling in pancreatitis and stromal fibrosis, chronic obstructive pulmonary disease, pulmonary fibrosis, uterine fibroids, ovarian fibrosis, other fibrocystic formations, corneal fibrosis or other eye fibrosis, such as that resulting from corneal refraction surgery, fibrosis resulting from congestive heart failure and other post-ischemic conditions, abdominal adhesions, wide angle glaucoma trabeculotomy, and any combinations thereof.

42. The method of claim 41, said method further comprising administering to said mammal serum amyloid P (SAP) and/or fragments thereof, IL-12, Laminin-1, IgG aggregates, cross-linked IgG, cofactors of any of the above, and any combination thereof or a pharmaceutical composition comprising serum amyloid P (SAP) and/or fragments thereof, IL-12, Laminin-1, IgG aggregates, cross-linked IgG, cofactors of any of the above, or any combination thereof.

43. The method of claim 42, said fragments of SAP being selected from the group consisting of KERVGEYSLYIGRHKVTSKVIEKFP (SEQ ID NO:1), ILSAYQGTPLPA (SEQ ID NO:2), IRGYVIIKPLV (SEQ ID NO:3), and combinations thereof.

44. The method of claim 42, wherein said DDX3 inhibitor or composition thereof and serum amyloid P (SAP) and/or fragments thereof, IL-12, Laminin-1, IgG aggregates, cross-linked IgG, cofactors of any of the above, and any combination thereof or a pharmaceutical composition comprising serum amyloid P (SAP) and/or fragments thereof, IL-12, Laminin-1, IgG aggregates, cross-linked IgG, cofactors of any of the above, or any combination thereof are administered simultaneously, separately, or sequentially or are administered as a combined composition.

45. The method of claim 44, wherein said mammal is a human.

46. The method of claim 33, wherein said mammal is a human.

47. The method of claim 33, wherein the at least one DDX3 inhibitor is administered in an amount that ranges from about 0.1 mg/kg to about 100 mg/kg of a subject to be treated; about 1 mg/kg to about 75 mg/kg of a subject to be treated; about 1 mg/kg to about 50 mg/kg of a subject to be treated; about 10 mg/kg to about 100 mg/kg of a subject to be treated; about 10 mg/kg to about 75 mg/kg of a subject to be treated; about 10 mg/kg to about 50 mg/kg of a subject to be treated; about 1 mg/kg to about 75 mg/kg of a subject to be treated; about 10 mg/kg to about 30 mg/kg of a subject to be treated; about 15 mg/kg to about 30 mg/kg of a subject to be treated; about 15 mg/kg to about 25 mg/kg of a subject to be treated; or in an amount of about 20 mg/kg of a subject to be treated.

48. The method of claim 33, wherein the at least one DDX3 inhibitor is administered daily, every other day, two to six times per week, or once daily for two to 31 days per month.

49. The method of claim 33, wherein the at least one DDX3 inhibitor is administered orally, by injection, by intravenous infusion, topically, or by inhalation.

50. The method of claim 33, further comprising administering at least one DDX3 inhibitor in an amount and for a time sufficient to decrease the level or activity of NEU3.

51. The method of claim 48, wherein each dose subsequent to a first dose of said at least one DDX3 inhibitor that is administered to a subject in need of treatment is separated by at least one day and up to 31 days or more or by one to 12 months from the previous dose of said at least one DDX3 inhibitor.
